(19) United States
(12) Patent Application Publication
(10) Pub. No.: US 2011/0202049 A1
(43) Pub. Date: Aug. 18, 2011

(54) Small Gauge Ablation Probe For Glaucoma Surgery

(75) Inventors: Guangyao Jia, Irvine, CA (US); Glenn Robert Sussman, Laguna Niguel, CA (US)

(73) Assignee: Alcon Research, LTD., Fort Worth, TX (US)

(21) Appl. No.: 12/707,747

(22) Filed: Feb. 18, 2010

(51) Int. Cl. A61F 9/007; A61B 18/08

(52) U.S. Cl. 606/29

(57) ABSTRACT

An ablation probe for use in glaucoma surgery has a shaft, a heating element, and a pair of electrical connectors located on a distal end of the shaft. The pair of electrical connectors holds the heating element to the shaft. The heating element is sized and shaped to ablate a trabecular meshwork in a human eye. The ablation probe is advanced through a corneal incision until the heating element contacts the trabecular meshwork. An electrical current is passed through the heating element to ablate the trabecular meshwork.

### BACKGROUND OF THE INVENTION

The present invention relates to glaucoma surgery and more particularly to a method and device for performing glaucoma surgery using a small gauge ablation probe.

Glaucoma, a group of eye diseases affecting the retina and optic nerve, is one of the leading causes of blindness worldwide. Glaucoma results when the intraocular pressure (IOP) increases to pressures above normal for prolonged periods of time. IOP can increase due to an imbalance of the production of aqueous humor and the drainage of the aqueous humor. Left untreated, an elevated IOP causes irreversible damage the optic nerve and retinal fibers resulting in a progressive, permanent loss of vision.

The eye's ciliary body epithelium constantly produces aqueous humor, the clear fluid that fills the anterior chamber of the eye (the space between the cornea and iris). The aqueous humor flows out of the anterior chamber through the uveoscleral pathways, a complex drainage system. The delicate balance between the production and drainage of aqueous humor determines the eye's IOP.

Open angle (also called chronic open angle or primary open angle) is the most common type of glaucoma. With this type, even though the anterior structures of the eye appear normal, aqueous fluid builds within the anterior chamber, causing the IOP to become elevated. Left untreated, this may result in permanent damage of the optic nerve and retina. Eye drops are generally prescribed to lower the eye pressure. In some cases, surgery is performed if the IOP cannot be adequately controlled with medical therapy.

Only about 10% of the population suffers from acute angle closure glaucoma. Acute angle closure occurs because of an abnormality of the structures in the front of the eye. In most of these cases, the space between the iris and cornea is more narrow than normal, leaving a smaller channel for the aqueous to pass through. If the flow of aqueous becomes completely blocked, the IOP rises sharply, causing a sudden angle closure attack.

Secondary glaucoma occurs as a result of another disease or problem within the eye such as: inflammation, trauma, previous surgery, diabetes, tumor, and certain medications. For this type, both the glaucoma and the underlying problem must be treated.

FIG. 1 is a diagram of the front portion of an eye that helps to explain the processes of glaucoma. In FIG. 1, representations of the lens 110, cornea 120, iris 130, ciliary bodies 140, trabecular meshwork 150, and Schlemm's canal 160 are pictured. Anatomically, the anterior chamber of the eye includes the structures that cause glaucoma. Aqueous fluid is produced by the ciliary bodies 140 that lie beneath the iris 130 and adjacent to the lens 110 in the anterior chamber. This aqueous humor washes over the lens 110 and iris 130 and flows to the drainage system located in the angle of the anterior chamber. The angle of the anterior chamber, which extends circumferentially around the eye, contains structures that allow the aqueous humor to drain. The first structure, and the one most commonly implicated in glaucoma, is the trabecular meshwork 150. The trabecular meshwork 150 extends circumferentially around the anterior chamber in the angle. The trabecular meshwork 150 seems to act as a filter, limiting the outflow of aqueous humor and providing a back pressure producing the IOP. Schlemm's canal 160 is located beyond the trabecular meshwork 150. Schlemm's canal 160 has collector channels that allow aqueous humor to flow out of the anterior chamber. The two arrows in the anterior chamber of FIG. 1 show the flow of aqueous humor from the ciliary bodies 140, over the lens 110, over the iris 130, through the trabecular meshwork 150, and into Schlemm's canal 160 and its collector channels.

If the trabecular meshwork becomes malformed or malfunctions, the flow of aqueous humor out of the anterior chamber can be restricted resulting in an increased IOP. The trabecular meshwork may become clogged or inflamed resulting in a restriction on aqueous humor flow. The trabecular meshwork, thus, sometimes blocks the normal flow of aqueous humor into Schlemm's canal and its collector channels.

Surgical intervention is sometimes indicated for such a blockage. Numerous surgical procedures have been developed to either remove or bypass the trabecular meshwork. The trabecular meshwork can be surgically removed. Several stents or conduits are available that can be implanted through the trabecular meshwork in order to restore a pathway for aqueous humor flow. Each of these surgical procedures, however, has drawbacks. One approach that does not have the drawbacks of existing procedures involves using a small gauge ablation probe to remove trabecular meshwork tissue.

### SUMMARY OF THE INVENTION

In one embodiment consistent with the principles of the present invention, the present invention is an ablation probe with a shaft, a heating element, and a pair of electrical connectors located on a distal end of the shaft. The pair of electrical connectors holds the heating element to the shaft. The heating element is sized and shaped to ablate a trabecular meshwork in a human eye.

In another embodiment consistent with the principles of the present invention, the present invention is a method of treating glaucoma comprising providing an ablation probe with a shaft, a heating element, and a pair of electrical connectors located on a distal end of the shaft, the pair of electrical connectors holding the heating element to the shaft; wherein the heating element is sized and shaped to ablate a trabecular meshwork in a human eye; and passing an electrical current through the heating element to ablate an opening in a trabecular meshwork of a human eye. The method may also include advancing the distal end of the shaft through a corneal incision; and performing cataract surgery through the corneal incision; injecting viscoelastic material into an anterior chamber of the human eye.

It is to be understood that both the foregoing general description and the following detailed description are exemplary and explanatory only and are intended to provide further explanation of the invention as claimed. The following description, as well as the practice of the invention, set forth and suggest additional advantages and purposes of the invention.

### BRIEF DESCRIPTION OF THE DRAWINGS

The accompanying drawings, which are incorporated in and constitute a part of this specification, illustrate several embodiments of the invention and together with the description, serve to explain the principles of the invention.

FIG. 1 is a diagram of the front portion of an eye.

FIG. 2 is a perspective view of a small gauge ablation probe according to the principles of the present invention.

FIGS. 3 and 4 are views of a small gauge ablation probe as used in glaucoma surgery.

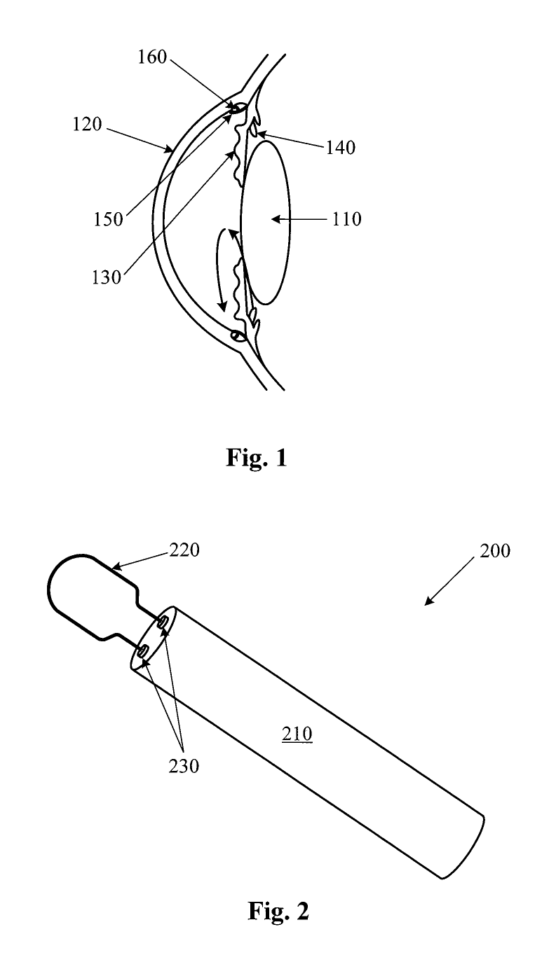

### DETAILED DESCRIPTION OF THE PREFERRED EMBODIMENTS

Reference is now made in detail to the exemplary embodiments of the invention, examples of which are illustrated in the accompanying drawings. Wherever possible, the same reference numbers are used throughout the drawings to refer to the same or like parts.

FIG. 2 is a perspective view of a small gauge ablation probe according to the principles of the present invention. In the example shown in FIG. 2, ablation probe 200 includes a shaft 210, a heating element 220, and a pair of electrical connectors 230. Shaft 210 holds heating element 220 and electrical connectors 230. Electrical connectors 230 couple heating element 220 to shaft 210. Heating element 220 is electrically coupled to wires that are located inside shaft 210. Shaft 210 can be connected to a surgical console via a pair of lead wires that provide electrical current to heating element 220. The surgical console can control the electrical current passed through heating element 220.

Shaft 210 may be made of any biocompatible material and can be of any desired shape. Typically, shaft 210 has a circular or elliptical cross section and a size of about 21 to 27 gauge. As such, shaft 210 is thin and suitable for insertion in a corneal incision used for cataract surgery.

Heating element 220 is typically a thin resistance wire that produces heat when a current is passed through it. Heating element is typically about 0.004 inches in diameter (or about 38 gauge), but may be 34 to 44 gauge in size. Heating element 220 may be made of any suitable material such as nichrome, kanthal, or copper nickel alloys, though other alloys or metals may be employed. While shown as a loop, heating element may be formed in any shape.

Electrical connectors 230 hold heating element 220 to shaft 210. Electrical connectors 230 couple heating element 220 to wires that provide current to heating element 220. Electrical connectors 230 may also serve as thermal insulators that protect shaft 210 from heat produced by heating element 220.

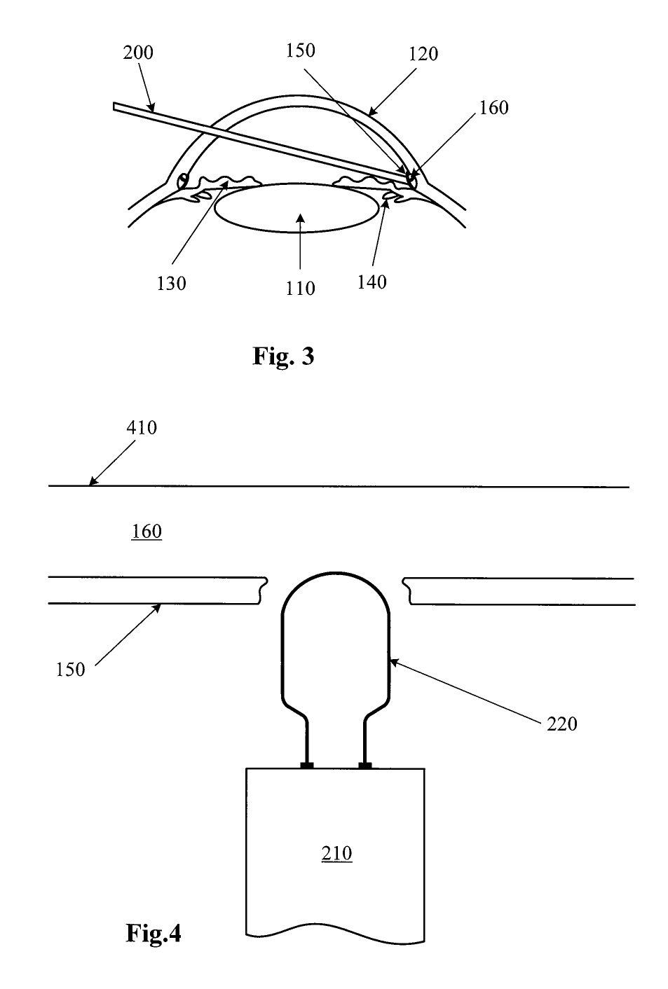

FIGS. 3 and 4 are views of a small gauge ablation probe 200 as used in glaucoma surgery. In FIG. 3, shaft 210 is inserted through a small incision in the cornea 120. The distal end of shaft 210 (the end that has heating element 220) is advanced through the angle to the trabecular meshwork 150. Current is passed through heating element 220 to produce heat that ablates the trabecular meshwork 150. Such a procedure can be performed in conjunction with cataract surgery.

A viscoelastic material may be injected into the anterior chamber during this procedure. The viscoelastic material absorbs heat produced by heating element 220 so that only the eye structure in direct contact with heating element 220 (i.e. the trabecular meshwork 150) is ablated leaving surrounding eye structures unharmed.

FIG. 4 is an exploded view of the location of the distal end of shaft 210 during the removal of the trabecular meshwork 150. In this position, the heating element 220 is located adjacent to and in contact with the trabecular meshwork 150. When current is applied to heating element 220, the trabecular meshwork 150 is ablated forming an opening between Schlemm's canal 160 and the anterior chamber. The back wall of Schlemm's canal 410 is unharmed. The process can be repeated as desired to form other openings in the trabecular meshwork 150. A series of pulses of electrical current can be passed through heating element 200 to produce a series of openings in the trabecular meshwork 150.

If desired, a second corneal incision opposite the first corneal incision can be made so that the outer heating element 220 can form openings in the trabecular meshwork 150 in a second arc of the angle. In this manner, either through one or two corneal incisions, a significant portion of the trabecular meshwork can be ablated by the ablation probe 200.

From the above, it may be appreciated that the present invention provides a system and methods for performing glaucoma surgery with a small gauge ablation probe. The present invention provides a small gauge ablation probe that ablates sections of the trabecular meshwork. Methods of using the probe are also disclosed. The present invention is illustrated herein by example, and various modifications may be made by a person of ordinary skill in the art.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. An ablation probe comprising:
a shaft;
a heating element; and
a pair of electrical connectors located on a distal end of the shaft, the pair of electrical connectors holding the heating element to the shaft;
wherein the heating element is sized and shaped to ablate a trabecular meshwork in a human eye.

2. The ablation probe of claim 1 wherein the heating element has a size of between 34 to 44 gauge.

3. The ablation probe of claim 1 wherein the shaft has a size of between 21 and 27 gauge.

4. A method of ablating the trabecular meshwork of a human eye, the method comprising:
providing an ablation probe with a shaft, a heating element, and a pair of electrical connectors located on a distal end of the shaft, the pair of electrical connectors holding the heating element to the shaft; wherein the heating element is sized and shaped to ablate a trabecular meshwork in a human eye; and
passing an electrical current through the heating element to ablate an opening in a trabecular meshwork of a human eye.

5. The method of claim 4 further comprising:
advancing the distal end of the shaft through a corneal incision

6. The method of claim 5 further comprising:
performing cataract surgery through the corneal incision.

7. The method of claim 4 further comprising:
injecting viscoelastic material into an anterior chamber of the human eye.

8. The method of claim 4 wherein passing an electrical current through the heating element further comprises passing a series of pulses of electrical current through the heating element.

9. The method of claim 4 further comprising:
ablating a second opening in the trabecular meshwork.
